(19) United States
(12) Patent Application Publication
(10) Pub. No.: US 2016/0015922 A1
(43) Pub. Date: Jan. 21, 2016

(54) LOWER MOUNT FLEXING ORAL-NASAL MASK

(71) Applicant: Koninklijke Philips N.V., Eindhoven (NL)

(72) Inventors: Lauren Patricia CHODKOWSKI, PITTSBURGH, PA (US); Peter Chi Fai HO, PITTSBURGH, PA (US)

(73) Assignee: Koninklijke Philips N.V., Eindhoven (NL)

(21) Appl. No.: 14/379,082

(22) PCT Filed: Feb. 18, 2013
(86) PCT No.: PCT/IB2013/051308

§ 371 Date: Aug. 15, 2014

Related U.S. Application Data
(60) Provisional application No. 61/603,641, filed on Feb. 27, 2012.

(51) Int. Cl. A61M 16/06

(52) U.S. Cl. CPC A61M 16/0622; A61M 16/0683

(57) ABSTRACT

The present invention provides a respiratory interface device including a mask having an upper, mask upper portion, a lower, mask lower portion, a flexible connecting member, and a patient contacting cushion. The flexible connecting member extends between, and is coupled to both, the mask upper portion and the mask lower portion. The flexible connecting member permits the mask upper portion and the mask lower portion to flex relative to one another. The patient contacting cushion is coupled to, and extends about, the mask upper portion, the mask lower portion, and the flexible connecting member. A brace assembly having a body with at least one mask coupling is provided. The brace is coupled to the mask upper portion and to the mask lower portion. The brace limits the outward rotation of the mask upper portion relative to the mask lower portion.

### CROSS-REFERENCE TO RELATED APPLICATIONS

This patent application claims the priority benefit under 35 U.S.C. §119(e) of U.S. Provisional Application No. 61/603,641 filed on Feb. 27, 2012, the contents of which are herein incorporated by reference.

### BACKGROUND OF THE INVENTION

1. Field of the Invention

The present invention relates to respiratory interface devices for transporting a gas to and/or from an airway of a user such as, but not limited to, a respiratory interface device, such as a mask, that includes a flexible faceplate having a mask upper portion, mask lower portion, and a substantially flexible connecting member extending therebetween, and, in particular, to a flexible respiratory interface device that is supported by a brace structured to bias the mask upper portion inwardly.

2. Description of the Related Art

A variety of respiratory masks are known which cover the areas surrounding the nose and/or mouth of a human user and that are designed to create an effective fit against the user's face. Typically, gases can be provided at a positive pressure within the mask for consumption by the user. The uses for such masks include high altitude breathing (aviation applications), swimming, mining, firefighting, and various medical diagnostic and therapeutic applications.

One requisite of many of these masks, particularly medical respiratory masks, is that they provide an effective fit against the user's face and that the mask contours with the user's face to limit or prevent leakage of the gas being supplied. A common type of mask incorporates a single-piece faceplate or a two-piece faceplate, having an upper portion (e.g., to cover the nasal portion of a human user's face) and lower portion (e.g., to cover the mouth portion of a human user's face) that are unitary or coupled together by a flexible member. See for example, U.S. Pat. Pub. 2011/0232647 which is incorporated by reference.

The respiratory masks that are known also have a sealing surface or cushion around the periphery of the mask to seal against the user's face. The cushion is typically attached to the faceplate. Such masks have performed well when the fit is good between the contours of the seal surface and the corresponding contours of the user's face. This may occur, for example, if the mask provides a good fit against the user's face and the mask contours with the user's face. If the fit is not good, there will be gaps in the mask-to-face interface resulting in gas leaking from the mask at the gaps. Considerable force will be required to compress the mask member to close the gaps and attain a satisfactory seal in those areas where the gaps occur. Typically, this required force will be provided by straps that are connected to the mask to securely fit the mask to the face of the user. Such force is undesirable because it produces high pressure points elsewhere on the face of the user where the mask contour is forcibly deformed against the face to conform to the user's facial contours. This will produce considerable user discomfort and possible skin irritation and breakdown anywhere the applied force exceeds the local perfusion pressure, which is the pressure that is sufficient to cut off surface blood flow.

One area of the cushion that is prone to being spaced from the user's face is the area about the bridge of the nose. That is, it is not uncommon to have a gap between the cushion and the user's face at the bridge of the nose or adjacent the bridge of the nose. When such a gap occurs, the user may adjust the straps by tightening or loosening the straps, as discussed above, or by moving the position of the straps relative to the user's face. Typically, the user would move the straps to a higher position on their face. This adjustment, however, places the straps closer to the user's eyes, which is generally considered to be uncomfortable. Alternatively, the respiratory interface device may be provided with additional straps so as to allow for better positioning of the respiratory interface device relative to the user's face. Additional straps, however, are also generally considered to be uncomfortable.

### SUMMARY OF THE INVENTION

One embodiment of the present invention provides a respiratory interface device including a mask having an upper, mask upper portion, a lower, mask lower portion, a flexible connecting member, and a patient contacting cushion. The flexible connecting member extends between, and is coupled to both, the mask upper portion and the mask lower portion. The flexible connecting member is structured to permit the mask upper portion and the mask lower portion to flex relative to one another. The patient contacting cushion is coupled to, and extends about, the mask upper portion, the mask lower portion, and the flexible connecting member. The respiratory interface device also includes a brace assembly having a body with at least one mask coupling. The brace is coupled to the mask upper portion and to the mask lower portion. The brace limits the outward rotation of the mask upper portion relative to the mask lower portion.

It is a further object of this invention to provide a method of using a respiratory interface device, the respiratory interface device including a mask having an upper, mask upper portion, a lower, mask lower portion, a flexible connecting member, and a patient contacting cushion, the flexible connecting member extending between, and coupled to both, the mask upper portion and the mask lower portion, the flexible connecting member structured to permit the mask upper portion and the mask lower portion to flex relative to one another, the patient contacting cushion coupled to, and extending about, the mask upper portion, the mask lower portion, and the flexible connecting member, a brace assembly having a body with at least one mask coupling, the brace being disengagably coupled to the mask upper portion and the mask lower portion, the at least one brace coupling includes at least one adjustable coupling structured to provide an inward bias to the mask upper portion, the method includes the steps of positioning the mask over the patient's nose and mouth thereby creating a generally continuous seal between the patient's face and the patient contacting cushion, and, adjusting the at least one adjustable coupling so as to create a more complete seal between the patient's face and the patient contacting cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is as side view of an exemplary embodiment of the respiratory interface device;

FIG. 2 is a front view of the exemplary embodiment of the respiratory interface device in FIG. 1;

FIG. 3 is a side view of another exemplary embodiment of the respiratory interface device;

FIG. 4 is a front view of another exemplary embodiment of the respiratory interface device;

FIG. 5 is a side view of the exemplary embodiment of the respiratory interface device in FIG. 4; and

FIG. 6 is a flow chart of the steps for the disclosed method.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, a “contact coupling” means that contacting parts are biased toward each other. That is, parts that merely contact each other without any bias do not form a “contact coupling.”

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall means that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. Further, as used herein, “inwardly” means in a direction toward the user's face and “outwardly” means in a direction away from the user's face.

As used herein, when discussing an element moving between two positions, e.g. a rod moving between a first position and a second position, “between” means at the first or at the second position, as well as any position therebetween.

As used herein, a “coupling” is one element of a coupling assembly. That is, a coupling assembly includes at least two elements, or couplings, that are structured to be coupled together. It is understood that the elements of a coupling assembly correspond to each other. For example, in a coupling assembly, if one coupling element is a bolt, the other coupling element is a nut. As a further example, in a coupling assembly, if one coupling element is a snap socket, the other coupling element is a snap plug. Further, it is understood that the two elements of a coupling assembly may not be described at the same time. It is further understood that coupling elements will be described by their location and that the other coupling element of the coupling assembly will have a similar description. For example, a mask may have a “first cheek coupling” and a brace may have a “first cheek coupling.” Thus, it is understood that when the brace and the mask are coupled, the “mask first cheek coupling” and the “brace first cheek coupling” are elements of a coupling assembly that correspond to each other as described above.

As used herein, the “bridge” of the nose is the area of the nose between the eyes. As used herein, the “dorsum” of the nose is the elongated area of the nose below the bridge and above the tip. The “dorsum” is, typically, aligned with the centerline of the face. The sides of the nose are not part of the dorsum. As used herein, “aligned” means “on the same line as” or “parallel to.”

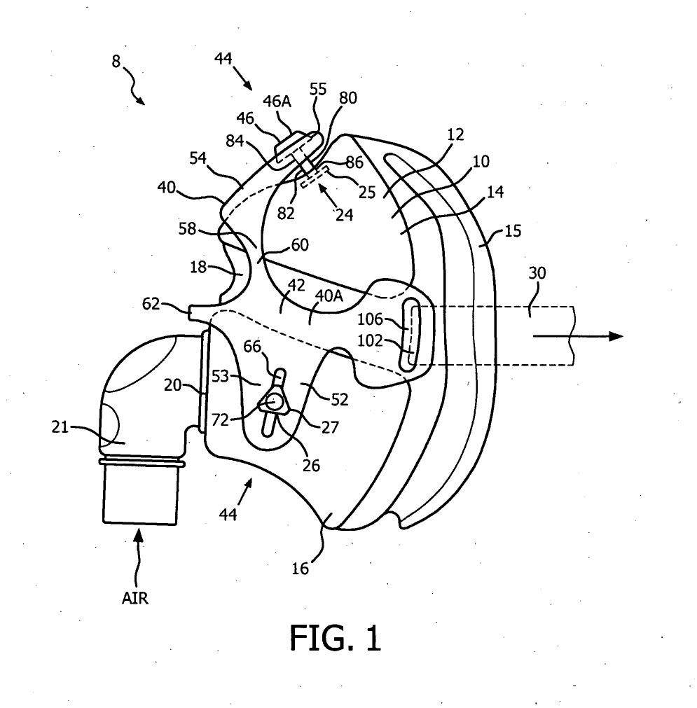

FIG. 1 is a side view of a respiratory interface device 8 according to an embodiment of the invention. Respiratory interface device 8 includes a respiratory mask 10 and a brace assembly 40. Mask 10 is coupled to a pressure generating system (not shown) via a patient circuit, as is conventionally known in the art. For purposes of the present invention, the pressure generating system is any device capable of generating a flow of breathing gas or providing gas at an elevated pressure. Examples of such pressure generating systems include a ventilator, CPAP device, or variable pressure device, e.g. an auto-titrating device, proportional assist ventilation (PAV®) device, proportional positive airway pressure (PPAP) device, C-Flex™. device, Bi-Flex™ device, or a BiPAP® device manufactured and distributed by Philips Respironics of Murrysville, Pa., in which the pressure provided to the patient varies with the patient's respiratory cycle so that a higher pressure is delivered during inspiration than during expiration, or other pressure support device.

Respiratory mask 10 includes a body 12 having upper portion 14 and lower portion 16. Body 12 further includes a substantially flexible connecting member 18 that extends between upper and lower portions 14 and 16, respectively. Connecting member 18 is structured to at least partially connect upper portion 14 to lower portion 16. Specifically, a first side of connecting member 18 is coupled to upper portion 14 and a second, opposite side, is coupled to lower portion 16. Upper and lower portions 14, 16 are typically, although not necessarily, generally rigid. That is, mask upper portion 14 may also be identified as a “first substantially rigid portion” and mask lower portion 16 may be identified as a “second substantially rigid portion.”

Connecting member 18 is a flexible, resilient member. In an exemplary embodiment of the present invention, connecting member 18 is made of, for example, a thermoplastic or thermoelastic material, including but not limited to an elastomer such as plastic, rubber, silicone, vinyl, foam, or any combination thereof. It is contemplated in the present invention that connecting member 18 can be formed in various shapes and geometries. In one embodiment, connecting member 18 provides a groove between upper and lower portions 14, 16. The groove (as shown in FIG. 1) can be structured to form a concave shape (e.g., an inward barrel shape). Alternatively, the groove can form a convex shape (e.g., an outward barrel shape). Connecting member 18 is structured to be flexible such that it can move in a hinge-like manner (as shown in FIG. 1). Such hinge-like movement allows upper portion 14 to flex or move independently or separately from and relative to lower portion 16. Thus, mask 10 can provide improved contouring to the user's face, and therefore result in an effective fit. The particular connecting member 18 shown in FIG. 1 is not meant to be limiting and it should be understood that the present invention contemplates a variety of different configurations for the flexible connecting member which are described in more detail elsewhere herein. Thus, various flexible connecting members may be substituted for connecting member 18.

In the embodiment as shown in FIG. 1, lower portion 16 also defines lower opening 20. Lower opening 20 can function as a gas inlet. In the embodiment shown in FIG. 1, gas inlet (lower opening 20) can be coupled to a coupling device 21, such as a swivel conduit, for carrying gas such as air between mask 10 and an external gas source (not shown), such as a blower, or any other suitable device. It is contemplated that the external gas source can encompass, without limitation, any gas delivery or gas generation system capable of supplying gas for consumption by a user. Non-limiting examples of various gas delivery therapies can include but are not limited to continuous positive airway pressure (CPAP) therapy, auto-titration positive airway pressure therapy, and bi-level positive airway pressure therapy, as noted above. The particular coupling device 21 shown in FIG. 1 is not meant to be limiting and it should be understood that the present invention contemplates a variety of different coupling devices that could be attached, either permanently or selectively, to lower opening 20 to carry gas to or from mask 10. Thus, a variety of coupling devices (e.g., with or without swivels on one or both ends, and with or without an exhalation system formed integral to the device) may be substituted for coupling device 21.

Mask 10 is an oral/nasal masks structured to accommodate both the oral and nasal regions of the user's face. Upper portion 14 can accommodate the nasal region, and lower portion 16 can accommodate the oral region. Mask 10 also includes a patient contacting cushion 15. In an exemplary embodiment, cushion 15 is integrally connected to upper portion 14, lower portion 16, and connecting member 18. Cushion 15 is structured to extend toward the user's face and generally defines the depth of mask 10. In an exemplary embodiment, a portion of cushion 15 defines all or at least a portion of connecting member 18. Cushion 15 can be constructed of a wide variety of materials known in the art and can include but is not limited to those materials previously described as suitable materials for upper and lower portions 14, 16 and connecting member 18. The particular cushion 15 shown in FIG. 1 is not meant to be limiting and it should be understood that other types of cushion supports or sealing systems that extend between the frame member (e.g., a faceplate 13 shown in FIG. 1) and the user's face, may be substituted for cushion 15. For example, and without limitation, various cushion configurations can include a double flap cushion, a gel cushion, a gel cushion with a flap, an air-filled cushion, a cushion with a pleat, multiple cushions (e.g., one inside of the other). Further, it is to be understood that the cushion 15 can be detachable and removable from the frame member (e.g., faceplate 13 in FIG. 1).

Mask upper portion 14 includes a nose coupling 24. In the exemplary embodiment, shown in FIG. 1, mask nose coupling 24 is a socket 25 having a rigid planar member. Mask nose coupling 24 is structured to be engaged by a distal end of an adjustable rod 82, discussed below. Thus, mask nose coupling 24 may be a contact coupling. Mask lower portion 16 may include a first cheek coupling 26 and a second cheek coupling 28. Mask lower portion first cheek coupling 26 and mask lower portion second check coupling 28 may be any type of coupling element such as, but not limited to, a snap element, a bolt element, or a ratchet element. Further, the cheek coupling 26 may not be adjustable for the system to work, thus a rigid connection such as, but not limited to an adhesive (not shown) may be used. In an exemplary embodiment, shown in FIG. 1, first and second cheek couplings 26, 28 are adjustable couplings 27, 29.

Respiratory interface device 8 further includes a strap 30. Strap 30 may be directly coupled to either mask 10 or brace assembly 40. Strap 30 may be a tension member or an elastic member. Strap 30 may include an adjustment device, such as, but not limited to, a buckle or a hook-and-loop coupling (neither shown). Such an adjustment device is structured to adjust the relative length of strap 30. Strap 30 is sized to extend about the user's head.

It is understood that mask upper portion 14 may generally rotate relative to mask lower portion 16. It is further understood that when mask 10 is in use, i.e. disposed on a user's face and secured by strap 30, that mask upper portion 14 is rotated outwardly, relative to mask lower portion 16, due to engagement with the user's face. As described below, brace assembly 40 is structured to limit the outward rotation of mask upper portion 14 relative to mask lower portion 16. This limitation of the of the outward rotation of mask upper portion 14 relative to mask lower portion 16 is hereinafter described as applying an inward bias to mask upper portion 14. It is understood that this bias occurs when mask 10 is in use. That is, it is understood that when mask 10 is not in use, and mask upper portion 14 is not rotated outwardly relative to mask lower portion 16, brace assembly 40 does not bias mask upper portion 14, but does still limit the outward rotation of mask upper portion 14.

That is, brace assembly 40 is coupled to mask 10 and provides a substantially stationary point relative to mask lower portion 16. The substantially stationary point is disposed adjacent mask upper portion 14. In this configuration, and when a user places mask 10 on their face and causes mask upper portion 14 to rotate relative to mask lower portion 16, mask upper portion 14 engages brace assembly 40. That is, mask upper portion 14 engages, i.e. is biased against, the stationary point on the brace assembly 40. Thus, the substantially stationary point provides a counter bias to mask upper portion 14 thereby arresting the rotation of mask upper portion 14 relative to mask lower portion 16.

Brace assembly 40 has a body 42 with at least one mask coupling 44. In an exemplary embodiment, at least one mask coupling 44 is at least one adjustable coupling 46 structured to provide an inward bias to mask upper portion 14. In another exemplary embodiment, at least one mask coupling 44 includes a plurality of adjustable couplings 46.

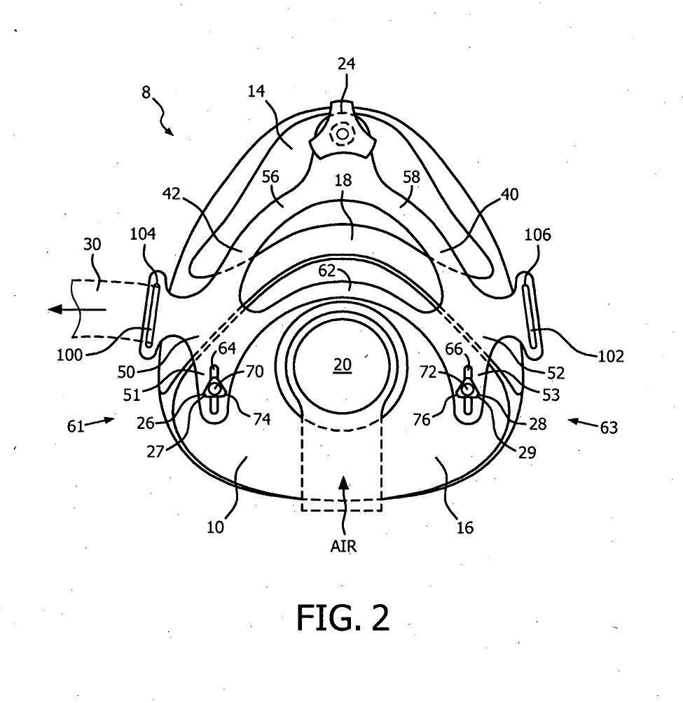

In an exemplary embodiment shown in FIGS. 1 and 2, a brace assembly 40A has a brace body 42 includes a right, first cheek portion 50, a left, second cheek portion 52, and an upwardly extending nose portion 54. In an exemplary embodiment, brace body first cheek portion 50, brace body second check portion 52 and brace body nose portion 54 include a generally planar area 51, 53, 55, respectively. First cheek portion planar area 51, second cheek portion planar area 53, and nose portion planar area 55 are structured to support a coupling element as described below. Between first cheek portion 50 and nose portion 54 is a first transition 56. Between second cheek portion 52 and nose portion 54 is a second transition 58. First and second transition portions 56, 58 may be flexure members 60. That is, flexure members 60 are structured to flex very slightly.

Flexure members 60 may be made from the same material as brace body 42, but have a smaller cross-sectional area. Flexure members 60 allow for a very slight flexing of brace body nose portion 54 relative to brace body first cheek portion 50 and brace body second cheek portion 52. This flexing may provide additional comfort to the user. The amount of flexure allowed, however, is very limited so as to not allow the flexing to affect the configuration of the mask 10 set by the various couplings as described below. Brace body 42 may also include a lateral portion 62. Lateral portion 62 extends between, and couples, first cheek portion 50 and second cheek portion 52. The brace body 42 may be a unitary body.

When in use, the plane of first cheek portion planar area 51 and the plane of second cheek portion planar area 53 extend generally parallel to the user's cheeks. Further, when in use, the plane of nose portion planar area 55 extends generally parallel to the dorsum of the user's nose. Thus, in broad terms, the plane of first check portion planar area 51 and the plane of second cheek portion planar area 53 extend generally vertically, and, the plane of nose portion planar area 55 extends generally at an angle relative to a vertical axis. That is, the dorsum of the user's nose is typically at an angle relative to a vertical axis.

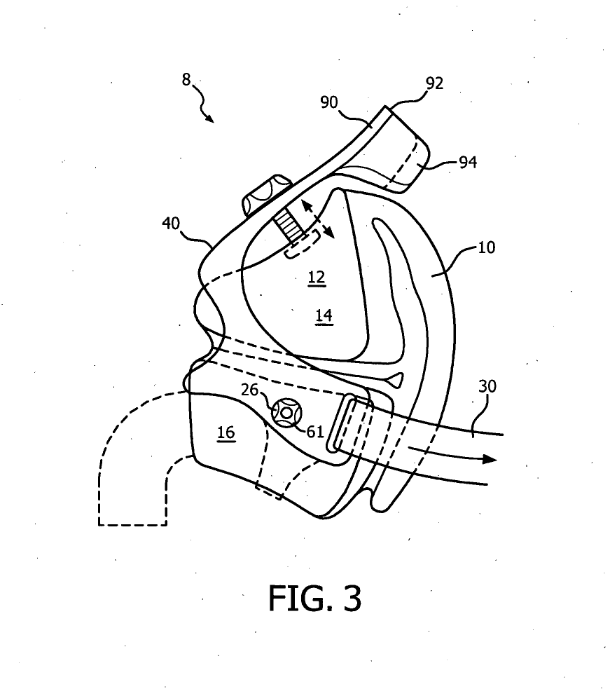

Brace body first cheek portion 50 has a first cheek coupling 61. Brace first cheek coupling 61 is positioned to engage mask lower portion first cheek coupling 26 when brace assembly 40 is coupled to mask 10. Brace body second cheek portion 52 has a second cheek coupling 63. Brace second cheek coupling 63 is positioned to engage mask lower portion second cheek coupling 28 when brace assembly 40 is coupled to mask 10. In one exemplary embodiment, mask lower portion first check coupling 26 (FIG. 3), mask lower portion second cheek coupling 28, brace first cheek coupling 61 (FIG. 3), and brace second cheek coupling 63 may be snap assemblies. In another exemplary embodiment, shown in FIGS. 1 and 2, mask lower portion first cheek coupling 26, mask lower portion second cheek coupling 28, brace first cheek coupling 61, and brace second cheek coupling 63 are adjustable coupling assemblies. For example, first cheek portion planar area 51 and second cheek portion planar area 53 may have an extended vertical length.

Further, in this exemplary embodiment, first check portion planar area 51 and second cheek portion planar area 53 have a generally vertical slot 64, 66 therethrough. Slots 64, 66 are hereinafter identified as “first cheek coupling vertical slot 64” and “second check coupling vertical slot 66.” Further, in this exemplary embodiment, mask lower portion first cheek coupling 26, mask lower portion second cheek coupling 28 are each threaded rods 70, 72. Thus, the corresponding coupling assembly element is a first cheek and a second cheek locking device 74, 76 such as, but not limited to, a knob having a threaded opening sized to correspond to threaded rods 70, 72.

When this exemplary embodiment of brace assembly 40A is coupled to mask 10, first cheek coupling threaded rod 70 is passed through brace body first cheek portion 50 first cheek coupling vertical slot 64, and, second cheek coupling threaded rod 72 is passed through brace body second cheek portion 52 second cheek coupling vertical slot 66. First cheek and second cheek locking devices 74, 76 are then coupled to the corresponding threaded rods 70, 72. The position of brace assembly 40A relative to mask lower portion 16 may be adjusted by repositioning threaded rods 70, 72 within first cheek coupling vertical slot 64 and second cheek coupling vertical slot 66. That is, first cheek coupling rod 70 may be selectively positioned between a first upper position and a second lower position within the brace first cheek coupling vertical slot 64, and, second cheek coupling rod 72 may be selectively positioned between a first upper position and a second lower position within the brace second cheek coupling vertical slot 66.

Further, the position of brace assembly 40A relative to mask lower portion 16 may be selectively locked by engaging first cheek and second check locking devices 74, 76. That is, first cheek and second cheek locking devices 74, 76 are tightened against first cheek portion planar area 51 and second cheek portion planar area 53, respectively. As discussed below, a coupling assembly couples brace nose portion 54 to mask upper portion 14. Thus, in this exemplary embodiment, selectively positioning brace body 42 relative to mask lower portion 16 alters the relative angle between mask upper portion 14 and mask lower portion 16. That is, if threaded rods 70, 72 are disposed at the upper end of first cheek coupling vertical slot 64 and second cheek coupling vertical slot 66, then brace nose portion 54 is spaced from mask upper portion 14 and mask upper portion 14 may rotate, relative to mask lower portion 16, away from the user's face. Conversely, if threaded rods 70, 72 are disposed at the lower end of vertical slots 64, 66, then brace nose portion 54 is closer to mask upper portion 14 and mask upper portion 14 may rotate relative to mask lower portion 16, but the degree of rotation is less than when threaded rods 70, 72 are disposed at the upper end of vertical slots 64, 66. Thus, selectively positioning threaded rods 70, 72 in first cheek coupling vertical slot 64 and second cheek Coupling vertical slot 66 limits the degree of outward rotation of mask upper portion 14 relative to mask lower portion 16. This, in turn, effects how cushion 15 engages and seals against user's face.

In an exemplary embodiment shown in FIG. 1, nose portion 54 includes at least one adjustable coupling 46 that is a brace nose coupling 80. Brace nose coupling 80 is positioned to engage mask nose coupling 24 when brace assembly 40A is coupled to mask 10. In this embodiment, brace nose coupling 80 includes a threaded rod 82 and a threaded opening 84 through nose portion 54, and in one exemplary embodiment, through nose portion planar area 55. Thus, in this exemplary embodiment, brace nose coupling 80 is an adjustable coupling 46A. Threaded opening 84 is sized to correspond to threaded rod 82, hereinafter “brace nose coupling rod 82.” Thus, brace nose coupling rod 82 may be installed in threaded opening 84 and adjusted axially. As nose portion planar area 55 extends in a plane generally parallel to the dorsum of user's nose, threaded rod 82 moves in a direction generally perpendicular to the dorsum of the patient's nose. Brace nose coupling rod 82 has a distal end 86. Brace nose coupling rod distal end 86 is disposed on the inner side of brace assembly 40A. Thus, brace nose coupling rod distal end 86 is structured to engage mask upper portion 14.

Brace nose coupling rod distal end 86 may be disposed in nose coupling socket 25. Nose coupling socket 25 provides a rigid surface that resists wear and tear caused by the rotation of threaded rod 82. With the exception of movement while being adjusted, brace nose coupling rod distal end 86 acts as a substantially stationary point. Further, as brace nose coupling rod 82 is adjustable relative to brace body nose portion 54, brace nose coupling rod distal end 86 is structured to be selectively positioned relative to the brace body nose portion 54. The position of brace nose coupling rod distal end 86 relative to brace body nose portion 54 defines the limit of the outward rotation of mask upper portion 14 relative to mask lower portion 16. Stated alternatively, the position of brace nose coupling rod distal end 86 relative to brace body nose portion 54 determines the bias applied to mask upper portion 14.

In this configuration, brace nose coupling 80 is structured to apply an inward bias to mask upper portion 14. That is, when mask 10 is in position on a user's face, the user's face engages the mask upper portion 14 causing mask upper portion 14 to rotate outwardly relative to mask lower portion 16. The rotation of mask upper portion 14 relative to mask lower portion 16 is arrested by mask upper portion 14 engaging brace nose coupling rod distal end 86. Thus, brace nose coupling rod distal end 86 provides a counter-bias to mask upper portion 14. This counter-bias is hereinafter referred to as the bias of the brace assembly 40A or the bias of a brace coupling.

Rotation of brace nose coupling rod 82 causes brace nose coupling rod 82 to move generally perpendicular to the dorsum of the patient's nose. When brace nose coupling rod 82 is moved toward the dorsum of the patient's nose, brace nose coupling 80 applies a greater bias in a direction generally perpendicular to the dorsum of the patient's nose. That is, when brace nose coupling rod 82 is moved toward the dorsum of the patient's nose, brace nose coupling 80 applies a greater inward bias to mask upper portion 14 and in a direction generally perpendicular to the dorsum of the patient's nose. This bias effects how cushion 15 engages and seals against user's face. Thus, at least one adjustable coupling 46A is structured to provide a biasing force adjacent the bridge of the patient's nose and, more specifically to provide a biasing force to mask upper portion 14 in a direction generally perpendicular to the dorsum of the patient's nose.

As shown in FIG. 3, brace assembly 40A may further include an elongated upward extension 90. Upward extension 90 has a distal end 92. Upward extension 90 is sized, i.e. has a length, such that upward extension distal end 92 is positioned over the patient's nasal bridge. A pad 94 is disposed on the inner side of upward extension 92. Pad 94 is structured to rest upon the patient's nasal bridge. Use of pad 94 creates a more stable mask 10.

Each of first check portion 50 and second cheek portion 52 include a strap coupling 100, 102. As shown, strap couplings 100, 102 may be a simple, generally vertical slot 104, 106 through which strap 30 may be looped. Alternatively, strap couplings 100, 102 may be a rigid loop (not shown) coupled to each of first check portion 50 and second cheek portion 52, or, a clip bracket (not show) for straps having clips (not shown). Regardless of the type of strap coupling 100, 102 used, the location of the strap coupling 100, 102 on the first cheek portion 50 and second check portion 52 ensures that strap 30 will be positioned away from the user's eyes. It is further noted that brace assembly 40A is limited to two strap couplings 100, 102. This configuration is generally considered to be more comfortable.

It is noted that mask couplings 44 described in association with this exemplary embodiment of brace assembly 40A are disengageable couplings 44. Thus, brace assembly 40A may be removed from mask 10. That is, brace assembly 40A is disengagably coupled to mask upper portion 14 and mask lower portion 16. In this configuration, the user may utilize brace assembly 40A when needed, and remove brace assembly 40A when not needed.

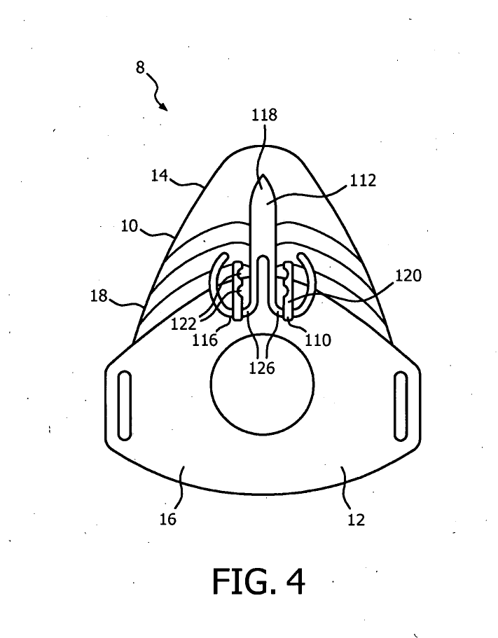

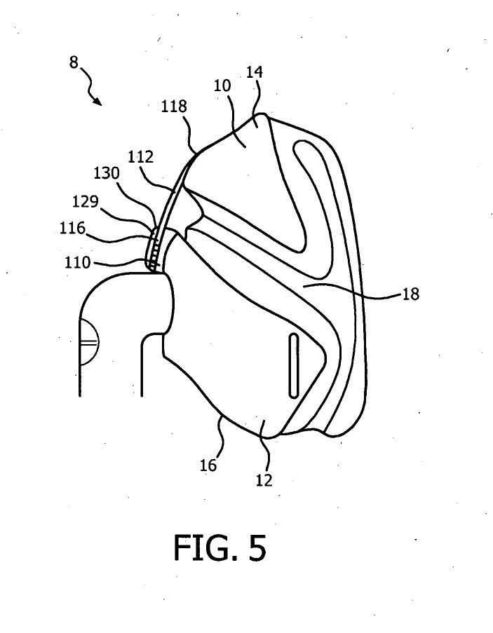

In another exemplary embodiment, shown in FIG. 4, brace assembly 40B includes a mounting 110 and a movable brace member 112. Mounting 110 is coupled, and, in an exemplary embodiment, fixed to one of mask upper portion 14 or mask lower portion 16. Movable brace member 112 is movably coupled to mounting 110 and extends toward, and engages the other of mask upper portion 14 or mask lower portion 16. As shown in FIG. 5, mounting 110 is disposed on lower mask portion 16 and movable brace member 112 extends toward, and engages mask upper portion 14. The following description shall address this configuration, but it is understood that position of mounting 110 and movable brace member 112 are reversible.

Movable brace member 112 has a fixed length and is substantially rigid. Movable brace member 112 has a proximal end 116 and a distal end 118. Movable brace member proximal end 116 is movably coupled to mounting 110. Movable brace member distal end 118 engages mask upper portion 14 in a contact coupling. Mask upper portion 14 may include a rigid plate (not shown) or similar structured that resists wear and tear due to engagement with movable brace member 112. Movable brace member 112 is generally aligned with, and movable in a direction generally aligned with, the centerline of the patient's face. Movable brace member 112 is movable between a first position and a second position. Mounting 110 is structured to selectively lock movable brace member 112 in any position between the first position and the second position. In this exemplary embodiment, the contact coupling between movable brace member distal end 118 and mask upper portion 14 is the adjustable coupling 46B. Adjustable coupling 46B is structured to provide a biasing force adjacent the bridge of the patient's nose and, more specifically to provide a biasing force to mask upper portion 14 in a direction generally perpendicular to the dorsum of the patient's nose.

In another embodiment, not shown, movable brace member distal end 118 may be disposed in a pocket (not shown) on mask upper portion 14 or may otherwise be fixed to mask upper portion 14. In this configuration, movable brace member 112 engaging mask upper portion 14, biases mask upper portion 14 inwardly. In the other configuration, wherein mounting 110 is coupled to mask upper portion 14, mounting 110 biases mask upper portion 14 inwardly. As noted above, the bias of brace assembly 40B is a counter-bias resulting from the bias of the user's face on mask upper portion 14. As before, this counter-bias shall be identified as the bias of the brace assembly 40B and its elements.

In an exemplary embodiment, mounting 110 includes a rack 120 having a plurality of stops 122. Mounting rack stops 122 may be slots 124 in between sets of parallel teeth (as shown), detents (not shown), aligned openings on parallel sidewalls (not shown), or any similar device. Mounting rack stops 122 are generally evenly spaced and generally in a line. Movable brace member proximal end 116 is structured to engage mounting rack stops 122. That is, if mounting rack stops 122 are slots 124, as shown, movable brace member proximal end 116 includes a lateral extensions 126 structured to fit within slots 124.

If mounting rack stops 122 are detents, movable brace member proximal end 116 includes a ball (not shown) or similar construct structured to engage the detents. If mounting rack stops 122 are opposed openings, movable brace member proximal end 116 includes opposed flexible tabs (not shown) as on a backpack quick-release buckle, or a similar construct structured to engage the openings. Mounting 110 may further include a locking device 128 structured to maintain movable brace member proximal end 116 at a selected mounting rack stop 122. Locking device 128 may be a cover 130 that extends over slots 124, as shown. In this exemplary embodiment, strap couplings 100, 102 are disposed on mask lower portion 16 and are disposed adjacent the right side and left side of mask lower portion 16.

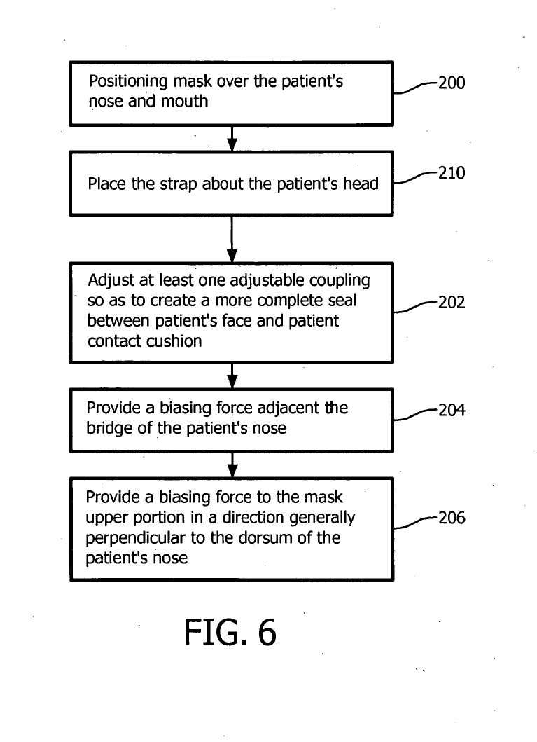

As shown in FIG. 6, a method of utilizing respiratory interface device 8 includes the steps of positioning 200 mask 10 over the patient's nose and mouth thereby creating a generally continuous seal between the patient's face and patient contacting cushion 15. Then, adjusting 202 at least one adjustable coupling 46 so as to create a more complete seal between the patient's face and patient contacting cushion 15. Generally, cushion 15 will create a generally continuous seal against the user's mouth and cheeks, but the seal may have gaps, or be loose, around the bridge of the user's nose. It is understood that by adjusting 202 at least one adjustable coupling 46, such gaps are reduced or eliminated. Thus, as used herein, a “more complete” seal is one that extends even slightly further than a generally continuous seal or is tighter than a generally continuous seal.

As noted above, at least one adjustable coupling 46, 46A, 46B is structured to provide a biasing force adjacent the bridge of the patient's nose and, more specifically, to provide a biasing force to the mask upper portion in a direction generally perpendicular to the dorsum of the patient's nose. Thus, the step of adjusting 202 at least one adjustable coupling 46 so as to create a more complete seal between the patient's face and patient contacting cushion 15 includes the step of providing 204 a biasing force adjacent the bridge of the patient's nose and, more specifically providing 206 a biasing force to the mask upper portion in a direction generally perpendicular to the dorsum of the patient's nose.

The step of positioning 200 mask 10 over the patient's nose and mouth thereby creating a generally continuous seal between the patient's face and patient contacting cushion 15 is typically accomplished by utilizing straps 30. Thus, the step of positioning 200 mask 10 over the patient's nose and mouth thereby creating a generally continuous seal between the patient's face and patient contacting cushion 15 includes the step of placing 210 the strap about the patient's head. Due to the brace assembly 40, 40A, 40B, and more specifically the adjustment of the adjustable coupling 46, 46A, 46B, cushion 15 may be adjusted without subsequently moving strap 30 relative to the patient's head.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by on and the same item of hardware. The word “a” or “an” preceding an element does not exclude the presence of a plurality of such elements. In any device claim enumerating several means, several of these means may be embodied by one and the same item of hardware The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A respiratory interface device comprising:
a mask including an upper portion, a lower portion, a flexible connecting member, and a patient contacting cushion, the flexible connecting member extending between, and coupled to both, the mask upper portion and the mask lower portion, the flexible connecting member structured to permit the mask upper portion and the mask lower portion to flex relative to one another, the patient contacting cushion coupled to, and extending about, the mask upper portion, the mask lower portion, and the flexible connecting member; and
a brace assembly having a body with at least one mask coupling, the brace being coupled to the mask upper portion and the mask lower portion, and wherein the outward rotation of the mask upper portion relative to the mask lower portion is limited.

2. The respiratory interface device of claim 1, wherein the at least one brace coupling includes at least one adjustable coupling structured to provide an inward bias to the mask upper portion.

3. The respiratory interface device of claim 2, wherein the brace at least one adjustable coupling includes a plurality of adjustable couplings.

4. The respiratory interface device of claim 2, wherein:
the brace body includes a right, first cheek portion, a left, second cheek portion, and an upwardly extending nose portion;
the mask lower portion having a first cheek coupling and a second cheek coupling;
the mask upper portion having a nose coupling;
the brace body first cheek portion having a first cheek coupling, the brace first cheek coupling positioned to engage the mask lower portion first cheek coupling when the brace is coupled to the mask;
the brace body second cheek portion having a second cheek coupling, the brace second cheek coupling positioned to engage the mask lower portion second cheek coupling when the brace is coupled to the mask; and
the brace body nose portion having a nose coupling, the brace nose coupling positioned to engage the mask upper portion nose coupling when the brace is coupled to the mask.

5. The respiratory interface device of claim 4, wherein:
the brace nose coupling is an adjustable coupling; and
the brace nose coupling is structured to apply a biasing force to the mask upper portion in a direction generally perpendicular to the dorsum of the patient's nose.

6. The respiratory interface device of claim 4, wherein:
the brace nose coupling includes a threaded rod having a distal end;
the brace nose coupling rod distal end structured to engage the mask upper portion; and
the brace nose coupling rod distal end structured to be selectively positioned relative to the brace body nose portion.

7. The respiratory interface device of claim 4, wherein:
the brace first cheek coupling includes a substantially vertical slot;
the brace second cheek coupling includes a substantially vertical slot;
the mask lower portion first cheek coupling includes a threaded rod, the rod sized to pass through the brace first cheek coupling vertical slot, and a first cheek locking device, the first cheek locking device structured to be selectively locked on the mask lower portion first cheek coupling threaded rod;
the mask lower portion second cheek coupling includes a threaded rod, the rod sized to pass through the brace second cheek coupling vertical slot, and a second cheek locking device, the second cheek locking device structured to be selectively locked on the mask lower portion second cheek coupling threaded rod;
wherein the mask lower portion first cheek coupling rod is disposed through the brace first cheek coupling vertical slot, wherein the first cheek coupling rod may be selectively positioned between a first upper position and a second lower position within the brace first cheek coupling vertical slot, and wherein the first cheek locking device is selectively coupled to the mask lower portion first cheek coupling rod at a location outside the brace body and biased against the brace body; and
wherein the mask lower portion second cheek coupling rod is disposed through the brace second cheek coupling vertical slot, wherein the second cheek coupling rod may be selectively positioned between a first upper position and a second lower position within the brace second cheek coupling vertical slot, and wherein the second cheek locking device is selectively coupled to the mask lower portion second cheek coupling rod at a location outside the brace body and biased against the brace body.

8. The respiratory interface device of claim 4, wherein:
the brace nose portion includes an upward extension, the upward extension having a distal end, the upward extension sized so that the upward extension distal end is positioned over the patient's nasal bridge; and
a pad disposed on the inner side of the upward extension, the pad being structured to rest upon the patient's nasal bridge.

9. The respiratory interface device of claim 4, wherein:
the brace first cheek portion includes a strap coupling; and
the brace second cheek portion includes a strap coupling.

10. The respiratory interface device of claim 9 wherein the brace has no more than two strap couplings.

11. The respiratory interface device of claim 4, wherein:
the brace body includes a first transition and a second transition, the first transition disposed between the first cheek portion and the nose portion, and, the second transition disposed between the second cheek portion and the nose portion; and
the first and second transition portions being flexure members.

12. The respiratory interface device of claim 2, wherein the brace assembly is disengagably coupled to the mask upper portion and the mask lower portion.

13. The respiratory interface device of claim 2, wherein:
the brace body includes a mounting and a movable brace member;
the mounting being coupled to one of the mask upper portion or the mask lower portion;
the movable brace member being movable coupled to the mounting and extending toward, and engaging, the other of the of the mask upper portion or the mask lower portion; and
wherein the mounting coupled to the mask upper portion, or, the movable brace member engaging the mask upper portion, biases the mask upper portion inwardly.

14. The respiratory interface device of claim 13, wherein:
the movable brace member is generally aligned with, and movable in a direction generally aligned with, the centerline of the patient's face;
the movable brace member being movable between a first position and a second position; and
the mounting structured to selectively lock the movable brace member in any position between the first position and the second position.

15. The respiratory interface device of claim 14, wherein:
the mounting includes a rack having a plurality of stops, the stops being generally evenly spaced and generally in a line;
the movable brace member having a proximal end, the movable brace member proximal end structured to engage the mounting rack stops;
the mounting having a locking device structured to maintain the movable brace member proximal end at a selected mounting rack stop; and
whereby the movable brace member may be placed and maintained in a selected position relative to the mounting rack.

16. The respiratory interface device of claim 14, wherein the mask lower portion has a first strap coupling disposed adjacent the right side and a second strap coupling disposed adjacent the left side.

17. A method of using a respiratory interface device, the respiratory interface device including a mask having an upper portion, a lower portion, a flexible connecting member, and a patient contacting cushion, the flexible connecting member extending between, and coupled to both, the mask upper portion and the mask lower portion, the flexible connecting member structured to permit the mask upper portion and the mask lower portion to flex relative to one another, the patient contacting cushion coupled to, and extending about, the mask upper portion, the mask lower portion, and the flexible connecting member, a brace assembly having a body with at least one mask coupling, the brace being disengagably coupled to the mask upper portion and the mask lower portion, the at least one brace coupling includes at least one adjustable coupling structured to provide an inward bias to the mask upper portion, the method comprising:
positioning the mask over the patient's nose and mouth thereby creating a generally continuous seal between the patient's face and the patient contacting cushion; and
adjusting the at least one adjustable coupling so as to create a more complete seal between the patient's face and the patient contacting cushion.

18. The method of claim 17, wherein the step of adjusting the at least one adjustable coupling so as to create a more complete seal between the patient's face and the patient contacting cushion includes the step of:
providing a biasing force adjacent the bridge of the patient's nose.

19. The method of claim 17, wherein the step of adjusting the at least one adjustable coupling so as to create a more complete seal between the patient's face and the patient contacting cushion includes the step of:
providing a biasing force to the mask upper portion in a direction generally perpendicular to the dorsum of the patient's nose.

20. The method of claim 17, wherein one of either the brace or the mask lower portion has a first strap coupling disposed adjacent the right side and a second strap coupling disposed adjacent the left side, the respiratory interface device further including a strap structured to encircle the patient's head, the ends of the strap being coupled to the first and second strap couplings, and wherein the step of positioning the mask over the patient's nose and mouth thereby creating a generally continuous seal between the patient's face and the patient contacting cushion includes the step of:
placing the strap about the patient's head; and
wherein adjustment of the at least one adjustable coupling allows for adjustment of the patient contacting cushion without moving the strap relative to the patient's head.
